(19) United States
(12) Patent Application Publication
(10) Pub. No.: US 2026/0199031 A1
(43) Pub. Date: Jul. 16, 2026

(54) DEVICE, SYSTEM AND METHOD FOR PERFORMING MINIMALLY INVASIVE PROCEDURES

(71) Applicant: Matan SHABAT, Eilat (IL)

(72) Inventor: Matan SHABAT, Eilat (IL)

(21) Appl. No.: 19/136,941

(22) PCT Filed: Dec. 7, 2023
(86) PCT No.: PCT/IL2023/051257

§ 371 Date: Jun. 9, 2025

(30) Foreign Application Priority Data
Dec. 7, 2022 (IL) 298926

(51) Int. Cl. A61B 34/00; A61B 17/00; A61B 18/00; A61B 18/24; A61B 90/53; A61M 25/09

(52) U.S. Cl. CPC A61B 34/25; A61B 18/24; A61B 90/53; A61B 2017/0034; A61B 2018/00559; A61B 2034/742; A61M 2025/09183

(57) ABSTRACT

In some embodiments of the disclosure a device, system and method for improving minimally invasive medical procedures, particularly useful for Natural Orifice Translumenal Endoscopic Surgery (NOTE) procedures, the device comprises a body in the form of a handpiece, a display, at least one working channel, a movement module and an actuating module.

### FIELD OF THE INVENTION

The present invention relates to minimally invasive medical procedures and in particular, to such a device, system and method for improving such minimally invasive medical procedures.

### BACKGROUND OF THE INVENTION

Minimally invasive procedures are performed by introducing various medical devices into a patient through small access holes utilizing trocars and/or cannulas.

More recent developments have led to the use of natural openings and/or anatomical orifices of the patient to be used as a point of entry. This has allowed to reduce or eliminate the need for making such access holes by way of incisions. The use of natural opening for performing medical procedures is known in the art as Natural Orifice Translumenal Endoscopic Surgery NOTEST procedures (herein NOTES). Accordingly, surgical devices, such as an catheters, endoscopes, imaging devices may be introduced to the treatment region through the working channels formed by the natural anatomical orifice. Such procedures utilize natural body orifice such as the mouth, anus, urethra, or vagina using translumenal access techniques and devices.

### SUMMARY OF THE INVENTION

Embodiments of the present invention provide a device and system configured for use in a vaginal NOTE™M (herein “vNOTE”) procedures in general and that is particularly useful but not limited for performing a vNOTE hysterectomy procedure according to other embodiments of the present invention.

Embodiments of the present invention provide a handheld device configured for Natural Orifice Translumenal Endoscopic Surgery (NOTE™M) procedure, the device comprising a: a handle portion; a display; a movement module ; a working channel featuring at least one maneuverable workpiece; actuating controller module; an electronic module; and a sensor module.

In embodiments of the present invention, the NOTE device wherein the positioning and orientation of the maneuverable workpiece may be controlled with the movement module. Optionally the movement module may be provided in the form of a joystick.

In embodiments the movement module may be functionally associated and controllable with the display wherein the display may be a touch sensitive display. In embodiments the movement module may be disposed along the handle portion of the device.

In embodiments, the joystick may be disposed along an external surface of the handle portion.

In embodiments, the maneuverable workpiece may be functionally coupled with at least one of: suction source, vacuum source and/or negative pressure source.

In embodiments, the maneuverable workpiece may be functionally coupled with at least one electromagnetic energy source for example including but not limited to a light source, laser source, electronic field, magnetic field, electromagnetic wavelength, the like or a combination thereof.

In embodiments, the maneuverable workpiece may be provided in the form of a guidewire, a balloon catheter, a catheter, a sleeve, or the like.

In embodiments, the actuating controller module may be provided for controlling and/or actuating the delivery of at least one or more of: the electromagnetic energy source and/or the negative pressure source.

In embodiments, the actuating controller module may be provided in the form of a trigger. Optionally the trigger may be a two stage controllable trigger.

In embodiments, the trigger may be a multi-stage controllable trigger.

In embodiments, the workpiece preferably comprises a distal “working” end having a controllable central lumen that may be controllably opened and/or closed with the actuating controller module.

In embodiments, controllable central lumen of the workpiece provides for controlling the delivery of suctioning and/or vacuum to the distal end of the workpiece.

In embodiments, the workpiece comprises a distal “working” end featuring a peripheral edges comprising at least a portion of the movement module.

In embodiments, the workpiece comprises a distal “working” end featuring a peripheral edges comprising optical fiber.

In embodiments, the workpiece comprises a peripheral edge along its length featuring at least one optical fiber.

In embodiments, the workpiece comprises a peripheral edge along its length featuring at least one effector of the movement module.

In embodiments, the effectors of the movement module may be provided in the form of steerable guidewire or pull wires or steerable wire or steerable coiled wire any combination thereof or the like.

In embodiments, the workpiece comprises a peripheral edge that may be embedded with a steerable wire along its length and wherein the guidewire is controllable with the movement module.

In embodiments, the steerable wire may be a steerable guidewire.

In embodiments, the workpiece features at least two steerable guidewires embedded along the length of the workpiece, and wherein the at least two steerable wires are embedded along the peripheral edge of the work oriented opposite one another and wherein at least one of the steerable guidewires is controllable with the movement module.

In embodiments, the workpiece comprises a flowing fluid flow channel.

In embodiments, the workpiece comprises a two way flowing fluid flow channel.

In embodiments, the fluid flow channel may be a flowing fluid delivery channel.

In embodiments, the fluid flow channel may be a flowing fluid evacuation channel.

In embodiments, the workpiece comprises at least two flowing fluid flow channel.

In embodiments, at least one of the at least two fluid flow channel may be a flowing fluid delivery channel.

In embodiments, at least one of the at least two fluid flow channel may be a flowing fluid evacuation channel.

In embodiments, the workpiece comprises a flowing fluid flow channel that may be disposed along the periphery of the workpiece.

In embodiments, the workpiece comprises a flowing fluid flow channel that may be disposed centrally.

Embodiments of the present invention provide a system comprising a handheld and operated NOTE device as previously described and an imaging device and/or sensor.

It should be emphasized that the following description discloses a NOTE device describing use in the vagina; however, the device, system and methods described herein are not limited to use with the vaginal anatomy and may be used with any anatomical area. For example, embodiments of the present invention may be used in different anatomic sites and/or different procedures for example including but not limited to trans-rectum colon surgery, laparoscopic surgery, intestinal surgery or examination where the point of entry is the anus, uterine surgery or examination where the point of entry is the bladder; and/or surgery or examination via the urethra and/or prostate surgery or examination via the urethra, and NOTES (Natural Orifice Transluminal Surgery)) are enclosed within the scope of the present invention.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagrams of an exemplary device and system according to embodiments of the present invention; and

FIG. 2A-C is a schematic illustrative diagram of an exemplary device according to an embodiment of the present invention; FIG. 2A shows perspective view detailing the working channel; FIG. 2B shows a further perspective view ; FIG. 2C shows a close up view of an optional embodiment of the distal end of a working channel; and

FIG. 3A is a schematic illustrative diagram showing an exemplary method for performing a vNOTE procedure according to an embodiment of the present invention; and

FIG. 3B is a flowchart depicting an exemplary method for performing a vNOTE procedure according to an embodiment of the present invention; and

FIG. 4A-C is a schematic illustrative diagram of an exemplary device according to an embodiment of the present invention; FIG. 4A shows a perspective view; FIG. 4B shows a side view; and FIG. 4C shows an in-use perspective view.

### DESCRIPTION OF THE PREFERRED EMBODIMENTS

The principles and operation of the present invention may be better understood with reference to the drawings and the accompanying description. The following figure reference labels are used throughout the description to refer to similarly functioning components are used throughout the specification hereinbelow.
50 system;
60 auxiliary device;
100 vNOTE handheld device;
102 device body;
102a handle portion;
102b upper portion;
102h hand brace housing;
102o proximal opening;
102f frame;
104 display;
105 working channel module;
105a maneuverable workpiece;
105b workpiece channels/functional unit channels;
105c workpiece central channel;
105d workpiece (distal) end;
105e workpiece maneuverable effector;
105f cover member;
105p peripheral edge;
105o optical fiber;
106 movement module;
108 actuating controller module;
108a first actuator;
108b second actuator;
110 electronics module;
111 user interface (UI);
112 power module;
114 controller and/or processor module;
116 communication module;
118 memory module;
120 sensor module;
122 temperature sensor;
124 pH sensor;
125 positional sensor;
126 imaging sensor;
128 flow sensor;
150 imaging device;

The use of natural opening for performing medical procedures is known in the art as Natural Orifice Translumenal Endoscopic Surgery NOTES™ procedures (herein NOTE). Accordingly, surgical devices, such as an catheters, endoscopes, imaging devices may be introduced to the treatment region through the working channels formed by the natural anatomical orifice. Such procedures utilize natural body orifice such as the mouth, anus, urethra, or vagina using translumenal access techniques and devices. Embodiments of the present invention are described with respect to a vaginal NOTE (herein “vNOTE”) device, system and method of use, however, the present invention is not limited to such anatomical site and/or procedures and/or use.

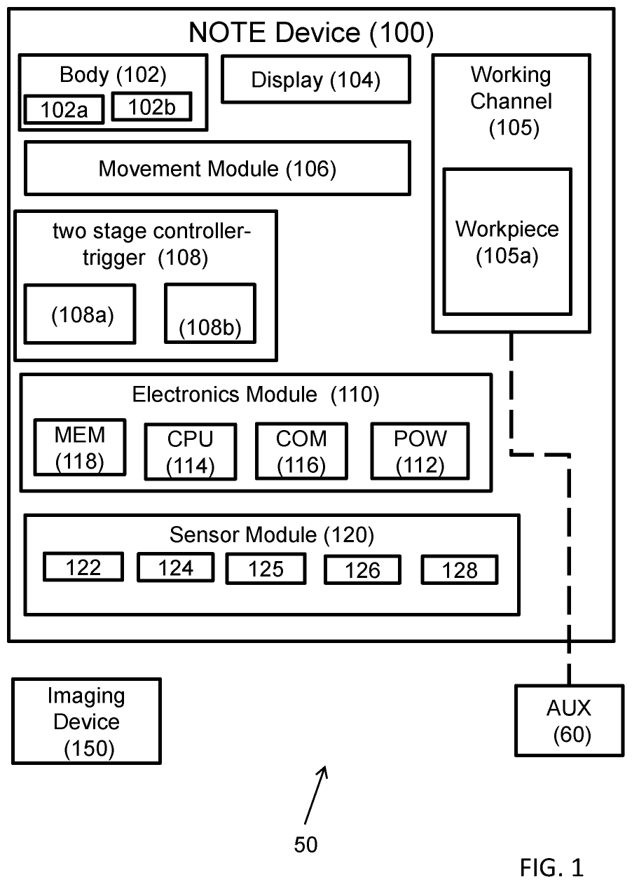

FIG. 1 shows a schematic block diagram of NOTE device 100 and more particularly a vaginal NOTE device that may be utilized and/or form system 50 thereof, according to embodiments of the present invention. In embodiments vNOTE device 100 may be configured for and/or used for performing a vNOTE hysterectomy according to embodiments of the present invention.

Embodiments of the present invention provide a handheld device 100 that is configured for Natural Orifice Translumenal Endoscopic Surgery (NOTE™) procedures. In embodiments device 100 features a working channel 105 and an associated maneuverable workpiece 105a. Preferably the maneuverable workpiece 105a provides for facilitating the introduction of tools for performing an endoscopic NOTE procedures for example including but not limited to suctioning, cutting, irrigating, excising, ablating, laser ablation, laser cutting, suturing, visualizing, marking, any combination thereof or the like endoscopic surgical procedures that is performed in the treated anatomy via the utilized natural orifice.

In embodiments the maneuverable workpiece 105a, is configured as a tool for performing any such endoscopic and/or remote surgical procedures as is necessary, most preferably in a minimally invasive manner.

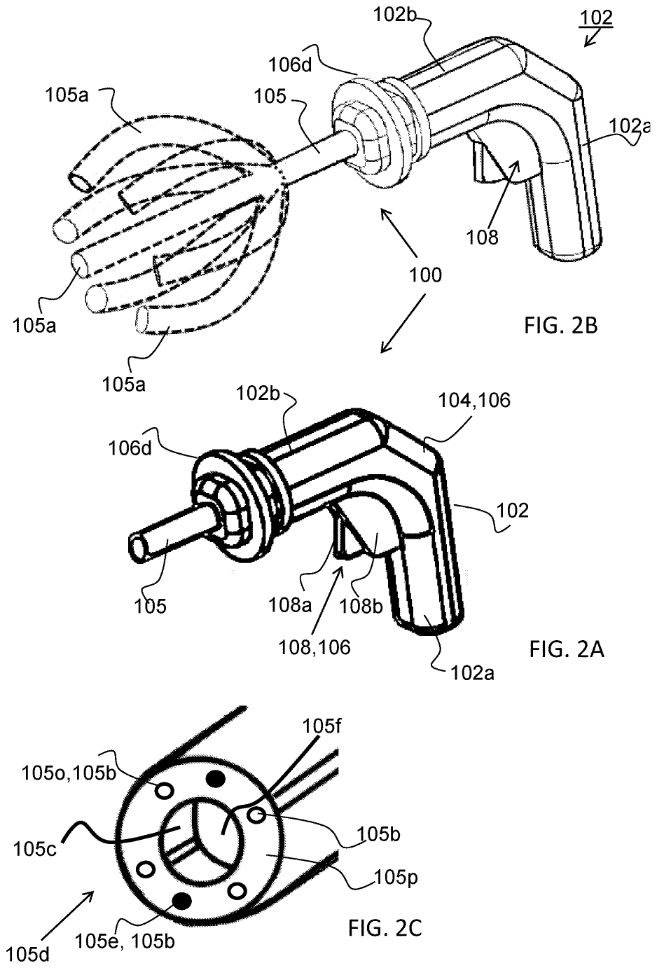

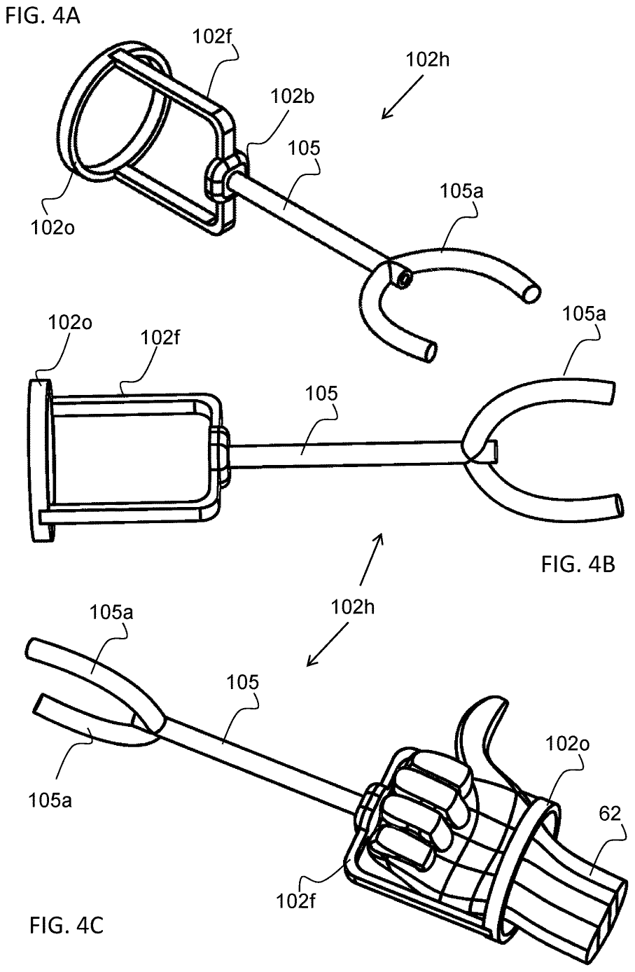

Device 100 comprises a device body 102 preferably provided in the form of a handpiece configuration, a non-limiting example of which may be seen in FIG. 2A-C and in FIG. 4A-C. The handpiece body 102 comprises a handle portion 102a and an upper portion 102b preferably featuring working channel 105, a non limiting example of which is depicted in FIG. 2A-C.

In embodiments body 102 is configured such that handle portion 102a is disposed perpendicular to upper portion 102b, (FIG. 2A-C).

In embodiments device 100 features a display 104 preferably configured for displaying and/or visually conveying the positional and/or orientation of at least one or more workpiece 105a associated with device 100.

In embodiments the working channel 105 features and/or is configured for accommodating and/or introducing least one or more maneuverable workpiece 105a. In some embodiments working channel 105 may be configured to accommodate at least two opposing maneuverable workpieces 105a.

In embodiments maneuverable workpiece 105a may assume optional orientations such that workpiece 105a is maneuverable and/or steerable and/or bendable to allow for performing optional surgical NOTE procedures, for example as shown in FIG. 2B.

In embodiments the maneuverable workpiece 105a may be provided in optional forms for example including but not limited to a guidewire, a balloon catheter, a catheter, a steerable catheter, a sleeve, a steerable sleeve, a multi-lumen steerable catheter, catheter comprising an optical fiber, flexible camera, imaging device the like or any combination thereof.

Optionally and preferably workpiece 105a is provided in the form of a steerable catheter and/or sleeve having a plurality of functional unit channels and/or workpiece channels 105b,c disposed along the length of workpiece 105a, as best seen in FIG. 2A-C.

In embodiments workpiece channels 105b may be arranged peripherally along the length of workpiece 105a.

In embodiments, workpiece channels 105c may be arranged centrally, therein forming a central channel 105c along the length of workpiece 105a.

Preferably each workpiece channel 105b,c may be utilized to perform an action with a dedicated functional unit at a distal end 105d of workpiece 105a while controlled at a proximal end with an actuator and/or controller disposed about a portion device body 102.

Optionally and preferably the functional units are rendered functional at least about a distal end 105d of workpiece 105a and wherein the functional unit's activity are preferably controlled at a proximal portion of workpiece 105a in associating with a controller disposed about portion of body 102 for example along handle 102a and/or upper portion 102b. Optionally a controller is associated with at least one of a movement module 106 and/or actuating controller module 108, so as to control the functional unit associated with the workpiece.

In embodiments, the maneuverable workpiece 105a comprises a distal end 105d, as best seen in FIG. 2C, having a controllable central channel 105c that may be controllably opened and/or closed with an actuating controller module 108 disposed about the device body. Optionally actuating controller module 108 may be provided in the form of a trigger like member.

Optionally, the controllable central lumen 105c may provide for controlling flowing fluid evacuation, for example suctioning, and/or fluid delivery, for example irrigation, at distal end 105d.

In embodiments central lumen 105c may be fit with a cover 105f that is controllably moved in order to open and/or close central lumen. Optionally, the cover 105f may be controlled with at least one member of the actuating controller module 108.

In embodiments, the distal end 105d may feature a peripheral edge 105p featuring at least one or more functional unit channels and/or workpiece channels 105b, as best seen in FIG. 2C. As previously described channels 105b are span the length of workpiece 105a and are controllable at a proximal end most preferably with a controller disposed about a portion of device body 102.

Optionally and preferably, individual channels 105b may be associated with a functional unit and/or function to render different functional at a portion of workpiece 105a. For example, a first channel 105b may be utilized to house and/or be integrated with an optical fiber 105o, as shown in FIG. 2C, to facilitate introducing light energy, for example in the form of a laser, at workpiece distal end 105d at the tissue treatment site. Most preferably the utilizing of optical fiber 105o is controllable with at least one control actuator of module 108, for example a trigger like actuator 108b, FIG. 2A-B, as may be utilized to controllably delivery electromagnetic energy in the form of laser to a tissue treatment site, wherein the laser source (not shown) is provided by an auxiliary device 60 optionally disposed external device 100 and coupled and/or associated with workpiece 105a about a proximal end thereof, but controlled with at least one or more actuator and/or controller disposed about device body 102, for example trigger 108b.

Optionally and preferably a dedicated workpiece channel 105b may be integrated with and/or feature a maneuverable effector 105e, as shown in FIG. 2C, that is preferably configured to provide workpiece 105a with controllable maneuverability rendering workpiece 105a steerable. For example, as shown in FIG. 2B, workpiece 105a may be steerable so as to assume various positions and/or orientations as shown in the optional positioning of workpiece 105a. Most preferably the orientation and positioning of workpiece 105a with maneuverable effectors 105e may be controllable with movement module 106.

In embodiments, optionally the steerable and/or maneuverable effector 105e may be provided in optional forms for example including but not limited to steerable guidewire, pull wires, steerable wire, steerable coiled wire, the like or any combination thereof. Most preferably the maneuverable effectors 105e is associated with movement module 106 configured for controlling the orientation or effectors 105e that in turn control the disposition and/or orientation of workpiece 105a to assume optional orientations and/or positions for example as shown in FIG. 2B.

In embodiments, the steerable workpiece 105a preferably comprise a peripheral edge that is embedded with a steerable wire 105e along its length and wherein the guidewire 105e is controllable with the movement module 106.

In embodiments, the workpiece 105a may feature at least two steerable guidewires 105e that are embedded along its length. Optionally the at least two steerable wires are embedded along the peripheral edge 105p and oriented opposite one another. Optionally and preferably at least one of the two embedded steerable guidewires is controllable with the movement module.

In embodiments, workpiece 105a may comprise a flowing fluid flow channel that is disposed centrally 105c, or peripherally 105b, for example as shown in FIG. 2C. In some embodiments, the fluid flow channel may be configured to be a two way flowing fluid flow channel capable of being a fluid delivery channel, for example for irrigation, or a fluid evacuation channel, for example for suctioning by the application of a negative pressure and/or vacuum.

In some embodiments, the workpiece 105 may comprise at least two flowing fluid flow channels, that may be disposed centrally 105c or peripherally 105b. Optionally, at least one of the at least two fluid flow channel may be a flowing fluid delivery channel. Optionally, at least one of the at least two fluid flow channel may be a flowing fluid evacuation channel.

In embodiments maneuverable workpiece 105a may preferably be maneuverable and/or controllable with a movement module 106. For example, the disposition and/or orientation of workpiece 105a is preferably controlled with module 106 such that workpiece may maneuvered and/or controlled as needed by a practitioner and/or user.

In some embodiments movement module 106 may be optionally integrated with a display module 104. For example, a display 104 in the form of a touch screen may be utilized allowing for maneuvering workpiece 105a by way of finger movements along a touch sensitive screen. Optionally such touch sensitive display 104 may be disposed about an upper portion 102b of body 102.

In embodiments the movement module 106 may be provided in the form of joystick and associated actuators configured for controlling the position and/or orientation of workpiece 105a. Optionally, the joystick may be disposed on at least one of the upper portion 102b of body 102. Optionally the joystick may be disposed on any portion of body 102, for example handle 102a and/or along the actuating controller module 108, a non limiting example of which is shown in FIG. 2A.

In embodiments, movement module 106 may comprise at least one or more actuator and/or motor and/or associated electronics to facilitate steerability of workpiece 105a.

In some embodiments movement module 106 may comprise and configured to be functional with movement dials 106d, for example as shown in FIG. 2A-B, wherein at least one or more dials may be provided to control the position and/or maneuver of channel 105 and/or workpiece 105a.

In some embodiments movement module 106 may comprise and configured to be functional with a wearable device for example in the form of a gloves and/or glasses and/or goggles to provide allow a user to a maneuver workpiece 105a with a wearable device. For example, a haptic glove may be utilized to control the movement and/or positioning of a workpiece based on movement of hand and/or finger. For example, wearable virtual reality glasses and/or goggles may be used to control the positioning of workpiece 105a by head and/or eye movement.

In embodiments device 100 preferably further comprises an actuating controller module 108, configured for facilitating control of any tool and/or auxiliary device that is associated device 100 and in particular workpiece 105a. For example, controller module 108 may be configured to provide for controlling suctioning, irrigation, applying a treatment for example tissue cutting, for applying a laser, fluid delivery, medicinal delivery, or the like.

In embodiments work controller module 108 may be provided in optional form for example including but not limited to one of: a trigger, button, dimmer butter, joystick, a compound trigger, dials, any combination thereof or the like.

In some embodiments, body 102 may be provided in the form of hand brace housing 102h, configured to receive a user's hand, a non limiting example of which is shown in FIG. 4A-C. Hand brace body 102h is shown in the form of a housing configured to receive a user's hand at a proximal opening 102o and featuring a frame 102f that extends distally from the proximal opening 102o.

Hand brace housing 102h allows a user to further manipulate working channels 105 and/or maneuverable workpiece 105a with additional degree of motion wherein the movement of the user's wrist may be used to further manipulate the positioning of work channel 105 and/or workpiece 105a.

In an optional embodiment, a non limiting example of which is shown in FIG. 4C, hand brace housing 102h may be used with an optional auxiliary device 60 provided in the form of a wearable haptic glove and/or motion controlled gloves 60h, to controllably maneuver working channels 105 and/or maneuverable workpiece 105a. Preferably wearable devices are in functional communication with device 100 to allow for the manipulation of the workpiece 105a and/or channel 105.

In embodiments device 100 comprises electronics and/or circuitry module 110 for rendering device 100 and system 50 functional.

In embodiments device 100 may comprise a sensor module 120 that is functionally associated with the electronics module 110.

In embodiments device 100 may comprise electronic module 110 comprising electronics circuitry, hardware, software to render device 100 functional and/or operational.

In embodiments electronics module 110 may comprise a plurality of optional sub-modules for example including but not limited to a power supply module 112, controller and/or processor module 114, user interface module 111, and memory module 118. In a preferred embodiments electronics module 110 may further comprise a communication module 116.

In embodiments electronics module 110 may be functionally associated with and/or comprise a sensor module 120.

In embodiments, User Interface (UI) module 111 may provide a user with means for interfacing with device 100 preferably via processor module 114. User interface 111 may be provided in the form of an activation button, audiovisual display, the like or any combination thereof. In embodiments, UI module 111 may be provided in optional forms for example including but not limited to buttons, dials, displays, alphanumeric display, touch screen, touch pad, buzzer, tactile pad, at least one light emitting diode (LED), at least one organic LED (OLED), speakers, microphone, or any combination thereof.

In embodiments processor module 114 provides the necessary processing hardware and/or software necessary to render device 100 functional. In embodiments controller and/or processor module 114 may provide for controlling any portion of device 100 and in particular maneuverable workpiece 105a and sensor module 120.

In embodiments power module 112 provides the necessary hardware and/or software to power device 100 therein rendering device 100 operational. Power module 112 may for example be provided in optional forms for example including but not limited to battery, rechargeable induction battery, induction coil, capacitors, super capacitors, inductors the like power source or any combination thereof.

In embodiments communication module 116 preferably provides the necessary hardware and/or software to facilitate communication for device 100 and in particular with optional auxiliary devices 60. Optionally communication module 116 may be utilized to communicate with an optional auxiliary devices 60. For example, an auxiliary device 60 may for example include but is not limited to a smartphone, mobile processing and communication device, imaging device, server, computer, healthcare service provider dedicated system, first respondent call center, health care call center, the like or any combination thereof.

In some embodiments communications module 116 may be utilize various communication protocols for example including but not limited to wireless communication, cellular communication, wired communication, near field communication, BLUETOOTH, ZIGBEE, optical communication, the like and/or any combination thereof.

In embodiments memory module 118 provides the necessary hardware and/or software to facilitate operation of device 100 by enabling storing and/or retrieving stored data and/or the like as is known in the art.

In embodiments sensor module 120 provides the necessary hardware and/or software to facilitate operations of at least one or more sensor(s) associated with device 100 to enable sensing various events in and around device 100 both internal and/or external to device 100. In some embodiments, for example, sensor module 120 may be configured to sense the environment external to device 100, for example, the vNOTE site. For example, sensor module may be utilized to provide imaging of the treatment site, measuring the temperature, measuring the pH, or the like conditions in and around the treatment site.

In embodiments sensor module 120 may comprise at least one or more sensor selected from the group consisting of: image sensor 126, temperature sensor 122, pH sensor 124, flow-meter 128, pulse oximeter, pressure sensor, acoustic sensor, microphone, positional sensor 125, infrared sensor, optical sensor, wavelength specific imaging sensor, the like or any combination thereof.

In embodiments sensor module 120 or portions thereof may be disposed along any portion of device 100, and in particular near the distal 105d and/or working end of body 102.

In embodiments image sensor 126 may be provided in optional forms, for example including but not limited to camera. In some embodiments image sensor may be provided in the form of ultrasound sensor, that may optionally be disposed along a portion of body 102. In some embodiments image sensor 126 may be provided in the form of wavelength specific imaging device such as an infrared optical sensor and infrared light source.

In embodiments an image sensor 126, optionally in the form of a camera, may be configured to provide image processing and/or analysis so as to infer and/or obtain parametric data relating to the targeted and/or treated tissue site. For example, such image processing analysis may provide to observe and/or determine at least one parameter associated with the targeted and/or tissue treatment site, for example including but not limited to at least one of: length, width, height, area, volume, shape, surface area, tissue type, color, the like or any combination thereof.

In some embodiments sensor module 120 may comprise at least one position sensor 125 configured to identify the position of a maneuverable workpiece 105a. In embodiments position sensor 125 may be provided in optional forms for example including but not limited to optical sensors, mechanical sensors, electromagnetic sensor, induction sensor, magnetic based sensors or the like sensor provided for determining and the relative position of workpiece 105a and in particular distal end 105d within the human anatomy.

In embodiments device 100 and in particular workpiece 105a may be functionally coupled and/or associated with optional auxiliary device 60 that are preferably utilized to provide a treatment, energy source, tool or the like to be utilized at the tissue treatment site and most preferably the distal end 105d of workpiece 105a.

In embodiments, optional auxiliary devices 60 that may for example include but is not limited to at least one of: suction source, vacuum source, negative fluid pressure source, positive fluid pressure source, light source, laser source, electromagnetic energy source, acoustic energy source, electric field source, magnetic field source, electromagnetic wavelength source, infrared energy source, any combination thereof or the like.

In embodiments, optional auxiliary devices 60 that are configured to render control of workpiece 105a may for example include but is not limited to at least one of: haptic gloves, motion controlled gloves, virtual reality goggles, virtual reality glasses, any combination thereof of the like.

In embodiments, the optional auxiliary device 60 may be controlled and/or actuated with actuating controller module 108. Optionally, the controller module 108 comprises controller(s) in optional form, for example including but not limited to a trigger, as best seen in FIG. 2A-B. Optionally, controller module may comprise a two stage controller for example in the form of a two stage controllable trigger have a first trigger 108a and a second trigger 108b. Optionally, the trigger may be a multi-stage controllable trigger.

Optionally a first trigger 108a may be utilized to control a first activity, for example application of a suctioning, while a second trigger 108b may be utilized a second activity, for example application of laser light exposure.

In embodiments device 100 may be utilized to form a system 50 comprising the device 100 as previously described with a medical imaging device 150 and/or at least one or more auxiliary device 60.

In embodiments system 50 may comprise device 100 that is in communication with a at least one or more auxiliary device 60. In embodiments auxiliary device 60 may be provided in optional forms for example including but not limited to a server, computer, smartphone, or the like processing and communication device capable of receiving and/or exchanging data from device 100 so as to process the data. Optionally communication between auxiliary device 60 and device 100 may be facilitated with communication module 116 utilizing any communication protocol for example including wireless, cellular, wired, near field, the like or any combination thereof.

In embodiments, an image device 150 may be utilized directly with device 100 and/or system 50 to visualize and communicate images of the treatment process and/or treatment substantially in real time. Optionally and preferably an image device 150 may optionally further provide for assessing parameters relating to the treatment site utilizing image processing software.

In some embodiments device 100 and/or portions thereof for example workpiece 105a may be configured to be a single use device.

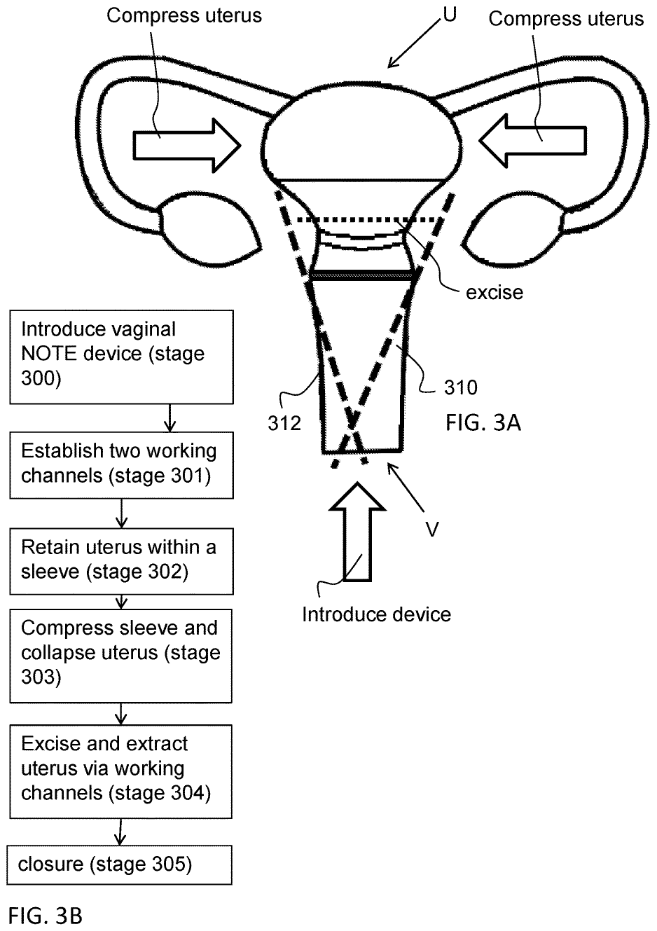

Now referring to FIG. 3A-B showing a flowchart of an exemplary method of utilizing device 100 in a vaginal NOTE procedure for performing a minimally invasive hysterectomy. FIG. 3A shows a schematic illustration of the uterus wherein a vaginal NOTE procedure is to be performed, via the vaginal opening, V. As shown, with the upward directional arrow at least a portion of NOTE device 100 may be introduced into the vaginal opening to two treatment channels 310, 312.

In embodiment of the method for performing a vNOTE hysterectomy, in a first stage 300 device 100 is introduced via the vagina ‘V’, and at stage 301 at least two work channels 310,312 are established. Optionally at least one of channels 310,312 may be utilized with device 100, while the second channel 312 may be utilized to introduce an imaging device 150 to facilitate visualization of the treatment site and/or procedure itself.

Next in stage 302, the uterus “U” is that is to be removed is retained with a sleeve, wherein most preferably containing sleeve was introduced with the assistance of device 100. For example, a retaining sleeve may be introduced via a steerable delivery workpiece 105a for example in the form of a steerable catheter to place the retaining sleeve about the uterus.

Next in stage 303, the contained uterus is compressed as shown by directional arrows in FIG. 3A so as to collapse the uterus and reduce any blood loss during the removal process.

Next in stage 304, the uterus is excised along at a selected tissue site, for example as shown in FIG. 3A. Most preferably cutting is provided with device 100, for example by utilizing a laser cutting tool introduced to the anatomy via channel 310 wherein workpiece 105a is steered such that distal end 105d is positioned opposite the excise tissue site line and laser cutting is performed via an embedded fiber optic 105o that is activated with trigger 108b while suctioning may be simultaneously provided via central channel 105c and is operated with trigger 108a, as shown in FIG. 2A.

Once the uterus is excised it is clamped and/or held with device 100 and removed through the vagina with device 100.

Lastly, the excision point is closed and the working channels 310,312 are closed.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely or the purpose of improving the intelligibility of the claims and are in no way limiting on the scope of the claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

What is claimed is:

1. A handheld device configured for Natural Orifice Translumenal Endoscopic Surgery (NOTE) procedures, the device comprising:
a. a device body (102) in the form of a handpiece;
b. a display (104);
c. a working channel (105) featuring at least one maneuverable workpiece (105a);
d. a movement module (106);
e. actuating controller module (108);
f. electronic module (110); and a
g. sensor module (120)

2. The device according to claim 1 wherein the position and orientation of the maneuverable workpiece is controlled with the movement module.

3. The device according to claim 1 wherein the movement module is provided in the form of a joystick.

4. The device according to claim 3 wherein the joystick is disposed on at least one of the upper portion of the body portion or the actuating controller module (108).

5. The device according to claim 1 wherein said movement module is functionally associated and controllable with said display wherein said display is a touch sensitive display.

6. The device according to claim 1 wherein said movement module is disposed along the handle portion of the device body.

7. The device according to claim 1 wherein said joystick is disposed along an external surface of the handle portion.

8. The device according to claim 1 wherein said maneuverable workpiece is functionally coupled with an auxiliary device (60).

9. The device according to claim 1 wherein the auxiliary device (60) is selected from at least one of: suction source, vacuum source, negative fluid pressure source, positive fluid pressure source, light source, laser source, electromagnetic energy source, acoustic energy source, electric field source, magnetic field source, electromagnetic wavelength source, infrared energy source, or any combination thereof.

10. The device according to claim 1 wherein said maneuverable workpiece is provided in the form selected from at least one of: a guidewire, a balloon catheter, a catheter, a steerable catheter, a sleeve, a steerable sleeve, a multi-lumen steerable catheter.

11. The device according to claim 1 wherein said actuating controller module is provides for controlling and/or actuating the use of at least one of said auxiliary device.

12. The device according to claim 11 wherein said actuating controller module is provided in the form of a trigger (108a,b).

13. The device according to claim 11 wherein said trigger is a two stage controllable trigger.

14. The device according to claim 11 wherein said trigger is a multi-stage controllable trigger.

15. The device according to claim 10 wherein said maneuverable workpiece comprises a distal end (105d) having a controllable central lumen that may be controllably opened and/or closed with said actuating controller module.

16. The device according to claim 15 wherein controllable central lumen of said workpiece provides for controlling the delivery of suctioning and/or vacuum to the distal end (105d) of said workpiece.

17. The device according to claim 15 wherein said workpiece comprises a distal end (105d) featuring a peripheral edge (105p) comprising at least a portion of said movement module.

18. The device according to claim 15 wherein said workpiece comprises a distal working end featuring a peripheral edges comprising an optical fiber (105o).

19. The device according to claim 15 wherein said workpiece comprises a peripheral edge along its length featuring at least one optical fiber.

20. The device according to claim 15 wherein said workpiece comprises a peripheral edge along its length featuring at least one maneuverable effector (105e) of the movement module.

21. The device according to claim 20 wherein said effector of the movement module is provided in the form of steerable guidewire or pull wires or steerable wire or steerable coiled wire any combination thereof or the like.

22. The device according to claim 20 wherein said workpiece comprises a peripheral edge that is embedded with a steerable wire along its length and wherein said guidewire is controllable with said movement module.

23. The device according to claim 20 wherein said steerable wire is a steerable guidewire.

24. The device according to claim 20 wherein said workpiece features at least two steerable guidewires embedded along the length of said workpiece, and wherein said at least two steerable wires are embedded along the peripheral edge of said work oriented opposite one another and wherein at least one of said steerable guidewires is controllable with said movement module.

25. The device according to claim 20 wherein said workpiece comprises a flowing fluid flow channel (105c).

26. The device according to claim 25 wherein said workpiece comprises a two way flowing fluid flow channel.

27. The device according to claim 25 wherein said fluid flow channel is a flowing fluid delivery channel.

28. The device according to claim 25 wherein said fluid flow channel is a flowing fluid evacuation channel.

29. The device according to claim 25 wherein said workpiece comprises at least two flowing fluid flow channel.

30. The device according to claim 29 wherein at least one of said at least two fluid flow channel is a flowing fluid delivery channel.

31. The device according to claim 30 wherein at least one of said at least two fluid flow channel is a flowing fluid evacuation channel.

32. The device according to claim 25 wherein said workpiece comprises a flowing fluid flow channel that is disposed along the periphery of said workpiece.

33. The device according to claim 25 wherein said workpiece comprises a flowing fluid flow channel that is disposed centrally.

34. The device according to claim 1 wherein the auxiliary device (60) is selected from at least one of: haptic gloves, motion controlled gloves, virtual reality goggles, virtual reality glasses, any combination thereof of the like.

35. The device of claim 1 wherein said movement module (106) comprises and is configured to be functional with a wearable device selected in the form of a haptic gloves and/or virtual reality glasses and/or virtual reality goggles.

36. The device of claim 1 wherein said body (102) is a provided in the form of a handpiece configuration including a handle portion (102a) and a tooling channel portion (102b) that are configured to be perpendicular to one another.

37. The device of claim 1 wherein said body (102) is provided in the form of hand brace housing (102h) configured to receive a user's hand.

38. The device of claim 37 wherein said hand brace housing (102h) features a proximal opening (102o) and a frame (102f) extending distally therefrom.

39. The device of claim 37 wherein said hand brace housing (102h) is configured to be used in conjunction with a glove (60h) in the form selected form a wearable motion controlling glove and/or haptic glove; wherein the glove is configured to facilitate to be in communication with device (100) so as to controllably maneuver the working channels (105) and/or the maneuverable workpiece (105a).

40. A system comprising the device according to any one of claim 1-39 and a medical imaging device (150).

41. The system of claim 40 further comprising at least one auxiliary device (60).

42. The system of claim 41 wherein the auxiliary device is at least one selected from: a server, a computer, a smartphone, a processing and communication device capable of receiving and/or exchanging data, a mobile processing and communication device, an imaging device, a healthcare service provider dedicated system, a first respondent call center, a health care call center, or any combination thereof.

43. The system of claim 41 wherein the auxiliary device is selected from at least one of: an energy source device, a tool, suction source, vacuum source, negative fluid pressure source, positive fluid pressure source, light source, laser energy source, electromagnetic energy source, acoustic energy source, electric field source, magnetic field source, electromagnetic wavelength source, infrared energy source, or any combination thereof.

44. The system of claim 41 wherein the auxiliary device (60) is selected from at least one of: haptic glove, motion controlled glove, virtual reality goggles, virtual reality glasses, any combination thereof of the like.

45. The device according to claim 25 wherein said flowing fluid flow channel (105c) comprises a cover member (105f) that is configured to be controllably moved in order to open and/or close the said flowing fluid flow channel (105c) with at least one member of the actuating controller module 108.
